(19) United States
(12) Patent Application Publication
(10) Pub. No.: US 2005/0042140 A1
(43) Pub. Date: Feb. 24, 2005

(54) Sensor for determining radiated energy and use thereof

(76) Inventor: Damian Fiolka, Oberkochen (DE)

Correspondence Address: SUGHRUE MION, PLLC, 2100 PENNSYLVANIA AVENUE, N.W., SUITE 800, WASHINGTON, DC, 20037, US

(21) Appl. No.: 10/482,775

(22) PCT Filed: Jul. 2, 2002
(86) PCT No.: PCT/EP02/07252

(30) Foreign Application Priority Data
Jul. 2, 2001 (DE) 101 31 918.5

(51) Int. Cl. G01N 33/00

(52) U.S. Cl. 422/91; 436/135; 250/379

(57) ABSTRACT

The invention relates to a sensor for determining the energy of radiation of a type that is capable of converting oxygen into ozone, and to a use of such a sensor. According to the invention, the sensor contains a measuring chamber (1) that can be transirradiated by the radiation and has a gas inlet (4) and a gas outlet (6), means (8) for feeding an oxygen containing gas (9) into the measuring chamber, via the gas inlet, and for discharging the gas via the gas outlet, one ozone sensor element (10) for measuring the ozone content of the gas (9a) located in the measuring chamber or discharged via the gas outlet, and evaluating means (12) for determining the radiant energy from the measured ozone content. The sensor can be used, for example, to determine radiant energy in an optical imaging system operating with the radiation. Use, for example, in microlithography projection exposure systems.

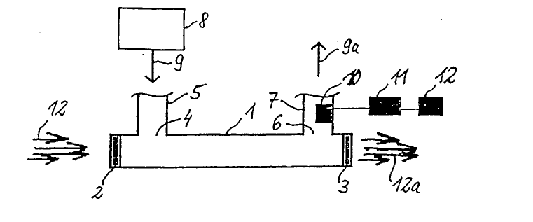

The invention relates to a sensor for determining the energy of radiation of a type that is capable of converting oxygen into ozone, and to a use of such a sensor.

Radiant energy sensors are in use in various designs and for various purposes, for example, in devices for controlling or regulating radiation sources of optical imaging systems, in order to set the radiant energy output by the radiation source to a desired, for example, constant value. Such a field of application is photolithographic projection exposure machines for imaging mask patterns on to resist-coated wafer surfaces in semiconductor technology that operates with UV radiation. UV radiation belongs to the type of radiation that converts oxygen into ozone when the radiation strikes an oxygen containing gas.

It is known to use photoelectric sensors operating with photodiodes for the abovementioned purpose of application in photolithographic projection exposure machines, in order to determine the energy of the radiation used for imaging and, on the basis thereof, to be able to set the radiant energy to a, for example, constant value, see patents U.S. Pat. Nos. 5.250.797, 5.728.495 and 6.141.081. These photoelectric sensors are used to determine the energy of UV radiation with wavelengths of, for example, 193 nm and 248 nm. The active surface of such sensors is, however, restricted to typically 2 mm×2 mm and is therefore relatively small. A further known type of sensors for determining the energy of electromagnetic radiation, specifically also in the UV region, are so-called pyrosensors. These are thermal sensors with a radiation-absorbing layer that heats up upon being irradiated and expands in the process. The expansion acts on a piezoelectric crystal which outputs an electric signal proportional to the thermal expansion.

Both in the case of the photoelectric sensors operating with photodiodes, and in the case of the pyrosensors, it is usual for the purpose of measurement for a fraction of the radiation produced by an associated radiation source to be coupled out as measuring radiation, for example, by means of a beam splitter, and for it to be fed to the sensor. This coupled out fraction of radiation is then no longer available for the actual useful radiation function.

The invention is based on the technical problem of providing a sensor of the type mentioned at the beginning and a use of the same, which permits a reliable determination of radiant energy in conjunction with a relatively slight loss of radiation, specifically also for UV radiation with low wavelengths of 157 nm, for example.

The invention solves this problem by providing a sensor having the features of claim 1, and a use of such as claimed in claim 6.

The sensor according to the invention contains a measuring chamber that can be transirradiated by the radiation and has a gas inlet and a gas outlet, means being provided for feeding an oxygen containing gas into the measuring chamber via the gas inlet, and for discharging the gas via the gas outlet. Moreover, the sensor contains one or more ozone sensor elements for measuring the ozone content of the gas located in the measuring chamber or discharged via the gas outlet. The radiant energy is determined by assigned evaluating means with the aid of the measured ozone content.

The sensor thus designed is suitable for determining the energy of radiation that, given the presence of oxygen, partially converts at least some of the latter into ozone. This ozone conversion is dependent in a defined way, for example, an empirically determinable way, on the radiant energy, for example, being proportional thereto. Consequently, when the measuring chamber is fed oxygen containing gas, the fed oxygen is converted at least partially into ozone by such radiation, coupled into the measuring chamber, the ozone content of the gas still located in the measuring chamber or discharged via the gas outlet being dependent on the radiant energy. Consequently, the evaluating means determine the targeted radiant energy from the measurement of the ozone content.

A substantial advantage of this sensor consists in that not all the radiation coupled into the measuring chamber is lost to the actual useful radiation function, but only that fraction which has contributed to the ozone conversion. The remaining fraction of measuring radiation can fulfill the envisaged useful function after coupling out from the measuring chamber.

In accordance with claim 2, in a development of the invention that is advantageous in terms of design, the measuring chamber is formed by a rectilinear measuring tube that can be traversed by the radiation in a longitudinal direction. The radiation can therefore traverse the measuring chamber rectilinearly without the need for radiation deflecting means. In addition, for a given measuring chamber volume, a relatively large transirradiation length results, and thus a higher degree of ozone formation, and this contributes to a high measuring sensitivity for a given quantity of oxygen in the measuring chamber.

In accordance with claim 3, in a further refinement of the invention that is advantageous in terms of design the gas inlet and the gas outlet are arranged at opposite end regions of the measuring chamber. This results in a correspondingly longer gas flow path through the measuring chamber, and this in turn contributes to an intensive interaction between the radiation coupled in and the oxygen contained in the fed gas, and thus to a high rate of ozone formation, and thus to the measuring sensitivity.

In a development of the invention as claimed in claim 4, the ozone sensor element is advantageously located in the region of a gas outlet or of a gas outlet line leading away therefrom such that said ozone sensor element does not disturb the radiation passing through the measuring chamber, and detects the ozone content of the gas in the region of the measuring chamber on the gas exit side, which gas contains all the ozone formed by the radiation.

In a development of the invention as claimed in claim 5, the gas feeding means are set up for variable setting of the feed rate and/or of the oxygen concentration of the oxygen containing gas. This can be used, for example, for the purpose of adjusting the measuring sensitivity of the sensor, and thereby of implementing a high measuring range dynamic for the sensor.

An advantageous use of the sensor according to the invention is provided, in accordance with claim 6, in an optical imaging system operating with the radiation. This can be, in particular, a photolithographic projection exposure machine. The radiant energy sensor can serve its purpose here, inside a controller or regulator, of detecting the energy, produced by an appropriate radiation source, of the radiation used, in particular UV radiation, in order, by means of a controller or regulator, to set the radiant energy to a specific value, for example to be able to keep it constant.

An advantageous embodiment of the invention is illustrated in the drawing and is described below.

The sole figure shows a schematic longitudinal sectional view of a sensor for determining radiant energy, for example, UV radiation.

The radiant energy sensor shown includes a measuring chamber formed by a rectilinear measuring tube 1. The measuring tube is sealed at both end faces by one radiation transparent window 2, 3 each, which does not absorb the radiation and is made from CaF₂, for example. Introduced at a slight distance from one end face into the lateral surface of the measuring tube 1 is a gas inlet 4 into which a gas inlet line 5 opens. Introduced into the lateral surface of the measuring tube 1 in a corresponding way at a slight distance from the other, opposite end face is a gas outlet 6 from which a gas outlet line 7 leads away. The gas inlet side 4, 5 of the measuring tube 1 is assigned conventional gas feed means 8, which are shown only schematically in the form of a block diagram and can be used to feed pure oxygen or another oxygen containing gas 9 into the gas feed line 5 at a feed rate and/or oxygen concentration that can be variably set. Positioned in the interior of the gas outlet line 7 is a conventional ozone sensor element 10 to whose electric measuring signal output an amplifier 11 is connected whose output signal is fed to an evaluating path 12 with an A/D converter and evaluating computer unit. Ozone sensor elements are in use, for example, in the form of so-called semiconductor sensors.

The sensor shown permits the determination of the energy of radiation of a type that is capable of converting oxygen into ozone by virtue of the fact that the radiation to be measured is guided through the measuring tube 1, in which oxygen-containing gas that has been fed is located, and the content, dependent on the radiant energy, of ozone formed is measured by the ozone sensor element 10.

During use, the radiation 12 to be measured, for example UV radiation with a wavelength of 157 nm, is coupled into the measuring tube 1 via one of its end faces by passing through the sealing window 2 there, and subsequently, traverses the rectilinear measuring tube 1 along its longitudinal direction and emerges from the measuring tube 1 again at the opposite end face by passing through the sealing window 3 there. At the same time, the gas feed means 8 feed the oxygen containing gas 9 at a desired, controllable feed rate and/or oxygen concentration to the measuring tube 1 via the gas inlet 4. The oxygen containing gas fed flows in the measuring tube 1 along the longitudinal direction thereof until it leaves said tube again via the gas outlet 6. Consequently, while the gas is traversing the measuring tube 1, the radiation 12 coupled in is in contact with the oxygen containing gas that is flowing through, as a result of which a fraction of the oxygen contained in the gas that is dependent on the radiant energy is converted into ozone. Consequently, the gas 9a discharged from the measuring tube 1 via the gas outlet 6 has an ozone content that is increased, as a function of the radiant energy, by comparison with the fed oxygen containing gas flow 9.

The ozone sensor element 10 detects this ozone content, that is to say the quantity of ozone formed per unit of time, and passes this information on to the amplifier 11 as an electric signal. The signal amplified by said amplifier is digitized in the evaluating part 12 of the A/D converter and then processed by the evaluating computer. The evaluating computer thereby determines the targeted radiant energy as a function of the measured ozone content with the aid of the functional dependence, known to it and for example empirically determinable, of said ozone content, of the energy of the radiation 12 traversing the measuring tube 1.

It is clear that the radiant energy sensor according to the invention and described above with the aid of a representative example, is suitable for the most varied fields of application in which the energy of an ozone forming radiation is to be detected, and has several specific advantages. An important field of application is the use of this radiant energy sensor in optical imaging systems for the purpose of detecting, and thereby monitoring the energy of the imaging radiation, and be able to set it to a respectively desired value. Specifically, the radiant energy sensor can be used in photolithographic projection exposure machines operating with UV radiation, in particular in their illuminating system. Systems with UV radiation of short wavelengths of, for example, 157 nm have recently been used in this case. There are otherwise few practicable radiant energy sensors for this radiation. The radiant energy sensor according to the invention permits adequately accurate generation of radiant energy precisely for such UV radiation of short wavelengths, since this radiation is absorbed by oxygen to the accompaniment of strong ozone formation, the ozone formation rate being proportional to the radiant energy.

An advantage of the radiant energy sensor according to the invention is its high dynamics with a logarithmic signal-to-noise ratio. The point is that its sensitivity can be regulated over a very wide measuring range by appropriate variation of the oxygen flow in the measuring tube 1. The oxygen flow can be set variably by the gas feed means 8, specifically by varying the gas feed rate and/or the oxygen concentration in the fed oxygen containing gas 9. Specifically, the radiant energy sensor according to the invention can be set to high sensitivity values by comparison with photoelectric sensors or pyrosensors of conventional type. The sensitivity of the ozone sensor element 10 is usually essentially constant in this case.

A further great advantage of the radiant energy sensor according to the invention consists in that it permits the determination of radiant energy in the traversing beam, that is to say the radiation 12a emerging from the sensor is available to the system for the purpose of fulfilling the actual useful function, in which case it is attenuated only slightly in its intensity by comparison with the radiation 12 coupled into the sensor, this attenuation being by that fraction which was absorbed by the oxygen in the sensor to the accompaniment of ozone formation. Depending on the case of application, the measuring chamber 1 can here be introduced directly into the beam path of the radiation to be measured, or a fraction of the radiation can be coupled out from the main beam path and then be led through the measuring chamber 1, and subsequently be coupled into the main beam path again.

The radiant energy sensor according to the invention is not subject to any aging in continuous operation, since freshly fed oxygen containing gas is always flowing through the sensor measuring chamber. The reaction time of the sensor is determined primarily by that of the ozone sensor element. For specific applications, as an alternative to this continuous flushing of the measuring chamber by gas, consideration is also given to feeding the oxygen containing gas only from time to time in a pulsating fashion during a respective measuring operation, or else not to undertake to feed any fresh oxygen containing gas during the measurement but to fill the measuring chamber initially with oxygen containing gas, and then to lead the radiation through the measuring chamber and thereafter to measure the ozone content of the gas in the measuring chamber or to flush the measuring chamber and to measure the ozone content of the gas driven out of the measuring chamber.

It goes without saying that various modifications of the radiant energy sensor shown are possible within the scope of the invention. Thus, depending on application, instead of the rectilinear measuring tube use may be made of any desired differently shaped measuring chamber that is traversed at least partially by the radiation whose energy is to be determined. Moreover, the position of the gas inlet and the gas outlet can be modified at will, and the coupling in and coupling out of the radiation, which is performed via the measuring tube end faces in the example shown, can likewise be provided at other locations of the measuring chamber. In this case, it is advantageous in general to have a longer gas transirradiation path referred to the measuring chamber volume, along which the radiation is in contact with the oxygen containing gas. Instead of being arranged in the gas outlet line, the ozone sensor element can also be arranged in the measuring chamber itself preferably in its region on the gas exit side. Moreover, several ozone sensor elements can be positioned at suitable locations if required.

1. A sensor for determining the energy of radiation of a type that is capable of converting oxygen into ozone, characterized by
a measuring chamber (1) that can be transirradiated by the radiation (12) and has a gas inlet (4) and a gas outlet (6),
means (8) for feeding an oxygen containing gas (9) into the measuring chamber, via the gas inlet, and for discharging the gas via the gas outlet,
at least one ozone sensor element (10) for measuring the ozone content of the gas (9a) located in the measuring chamber or discharged via the gas outlet, and
evaluating means (12) for determining the radiant energy from the measured ozone content.

2. The sensor as claimed in claim 1, further characterized in that the measuring chamber is formed by a rectilinear measuring tube (1) that can be traversed by the radiation (12) in a longitudinal direction.

3. The sensor as claimed in claim 1 or 2, further characterized in that the gas inlet (4) and the gas outlet (6) are located at opposite end regions of the measuring chamber (1).

4. The sensor as claimed in one of claims 1 to 3, further characterized in that the ozone sensor element (10) is arranged in the region of the gas outlet (6) or of a gas outlet line (7) leading away from said outlet.

5. The sensor as claimed in one of claims 1 to 4, further characterized in that the gas feeding means (8) are set up for variable setting of the feed rate and/or of the oxygen concentration of the oxygen containing gas.

6. A use of the sensor as claimed in one of claims 1 to 5, for determining the radiant energy in an optical imaging system operating with the radiation (12).
